(19) United States
(12) Patent Application Publication
(10) Pub. No.: US 2022/0314031 A1
(43) Pub. Date: Oct. 6, 2022

(54) ULTRASOUND APPARATUS AND RELATED METHODS OF USE

(71) Applicant: SMILESONICA, Inc., Edmonton (CA)

(72) Inventors: CRISTIAN SCURTESCU, Edmonton (CA); STEPHEN REBSTOCK, Edmonton (CA); CHRISTOPHER GERMAIN, Edmonton (CA)

(21) Appl. No.: 17/608,727

(22) PCT Filed: Jul. 15, 2020
(86) PCT No.: PCT/CA2020/050986

§ 371 Date: Nov. 3, 2021

Related U.S. Application Data
(60) Provisional application No. 62/874,774, filed on Jul. 16, 2019.

(51) Int. Cl. A61N 7/00; H01R 12/78; B06B 1/06

(52) U.S. Cl. CPC A61N 7/00; H01R 12/78; B06B 1/0622; B06B 1/0674; H01R 13/2442

(57) ABSTRACT

An improved ultrasound apparatus and methods of use are provided, the apparatus comprising at least one ultrasound transducer electrically connected to another electrical component by a flexible electrical connection. In some embodiments, the other electrical component is a printed circuit board. In some embodiments, the flexible electrical connection may allow vertical, horizontal and/or tilting displacement of the ultrasound transducer with respect to the flexible circuit board while maintaining electrical connectivity. In some embodiments, the flexible electrical connection is capable of temporarily disconnecting when an excessive deformation force is applied and self-reconnecting after the excessive deformation force is removed.

### CROSS-REFERENCE TO RELATED APPLICATION

The present application is a U.S. National Phase entry under 35 U.S.C. § 371 of PCT/CA2020/050986 filed on Jul. 15, 2020 entitled “ULTRASOUND APPARATUS AND RELATED METHODS OF USE,” which claims benefit of priority to U.S. Provisional Patent Application 62/874,774 entitled “ULTRASOUND APPARATUS AND RELATED METHOD” and filed Jul. 16, 2019, which is specifically incorporated by reference herein for all that it discloses or teaches.

### TECHNICAL FIELD

The present application relates to medical devices that emit ultrasound. More particularly, the present disclosure relates to improved intraoral ultrasound therapy devices and related methods of use.

### BACKGROUND

Intraoral therapy devices may be used to deliver therapeutic emissions such as ultrasound, light, heat, etc., to the roots of a patient's teeth, as well as the bone and tissues supporting and surrounding the roots of the teeth.

Conventional ultrasound therapy devices are typically comprised of at least one emitting element, such as an ultrasound transducer, for emitting at least one therapeutic emission. The transducer is connected to an electronics controller by two or more wires. For example, having regard to FIGS. 1A and 1B (PRIOR ART), in many known devices, transducer cables are rigidly connected to the transducer T by a permanent electrical connection C using electrical wires W and W′ attached to the transducer's electrodes E and E′ by soldering, conductive epoxy, or wire bonding. Unfortunately, such rigid connections between circuits may have poor reliability and may crack or break if the circuit is deformed beyond a certain threshold and/or is deformed repeatedly.

Improvements to known ultrasound therapy devices have consisted of using flexible arrays of ultrasound transducers. Arrays can be designed such that the strength of all internal electrical connections is higher than the maximum forces applied in the field. Unfortunately, even with flexible arrays, the electrical connections may be damaged if excessive force is applied or in the event of repeated long-term flexing of the arrays. Moreover, flexible arrays designed with strong internal electrical connections can also result in either a bulky array and/or an array with limited flexibility.

There remains a need for an improved intraoral ultrasound therapy device having flexible electrical connections capable of withstanding maximum forces applied to the connections, without cracking or breaking when the ultrasound transducer array is deformed.

### SUMMARY

According to embodiments, an improved ultrasound apparatus is provided, the apparatus having at least one ultrasound transducer, for emitting at least one ultrasound emission, a printed circuit board, and a flexible electrical connection between the at least one ultrasound transducer and the printed circuit board. In some embodiments, the apparatus comprises at least one array of ultrasound transducers, such as piezoceramic ultrasound transducers, each array of ultrasound transducers comprising at least eight ultrasound transducers.

In some embodiments, the present flexible electrical connection may comprise at least one spring contact, the spring contact having a mounting plate for securely affixing the contact to the flexible circuit board. The flexible electrical connection may further comprise at least one flexible circuit board finger for electrically coupling the at least one spring contact to the at least one ultrasound transducer. In some embodiments, the spring contact may comprise a cantilevered arm extending from the mounting plate for providing at least one electrical contact point.

In some embodiments, the present apparatus may be encapsulated within a housing formed of flexible material. The flexible material may comprise a biocompatible silicone elastomer or silicone rubber.

In some embodiments, the present apparatus may further comprise at least one layer of transducer backing material operably connecting the at least one transducer and the printed circuit board. The backing material may comprise a closed cell foam, or it may comprise nylon-based foam, polyurethane foam, or silicone foam. The backing material may be configured to form at least one aperture or slot for receiving and maintaining the at least one transducer.

According to embodiments, methods for providing ultrasound therapy are provided, the methods comprising providing at least one ultrasound transducer for emitting at least one ultrasound emission, providing a printed circuit board, connecting the at least one ultrasound transducer to the printed circuit board by a flexible electrical connection, and administering the at least one ultrasound emission to a patient.

In some embodiments, the flexible electrical connection provided may comprise at least one spring contact.

In some embodiments, the methods of providing the at least one ultrasound transducer may comprise providing a flexible array of ultrasound transducers. The array of ultrasound transducers may be encapsulated within a flexible housing material.

### BRIEF DESCRIPTIONS OF THE DRAWINGS

Embodiments of the present system will now be described by way of an example embodiment with reference to the accompanying simplified, diagrammatic, not-to-scale drawings. In the drawings:

FIG. 1A (PRIOR ART) is a top perspective view of an example prior art ultrasound transducer with a wrap-around electrode;

FIG. 1B (PRIOR ART) is top perspective view of an example prior art ultrasound transducer with one electrode on each side;

FIG. 2A (PRIOR ART) is a cross-sectional view of the ultrasound transducer of FIG. 1A packaged into a rigid housing;

FIG. 2B (PRIOR ART) is a cross-sectional view of the ultrasound transducer of FIG. 1B packaged into a rigid housing;

FIG. 3A is a front perspective view of the present intraoral ultrasound apparatus, according to some embodiments, the apparatus having upper and lower ultrasound emitting panels;

FIG. 3B is a top perspective view of the apparatus shown in FIG. 3A, where the upper mouthpiece transducer array is visible for ease of reference;

FIG. 4A is a side perspective view of an example spring contact, according to some embodiments;

FIG. 4B is a side perspective view of another example spring contact, according to some embodiments;

FIG. 4C is a side perspective view of yet another example spring contact, according to some embodiments;

FIG. 4D is a top perspective view of an example rigid printed circuit board having at least one example spring contact mounted thereon, according to embodiments;

FIG. 5 is a top perspective view of a flexible circuit board of the at least one upper panel of the apparatus shown in FIG. 3A (circle A), the panel having at least two of the spring contacts shown in FIG. 4A;

FIG. 6A is a top, perspective view of an example closed cell foam, with the adhesive exposed on the top surface, according to some embodiments;

FIG. 6B is a top perspective view of another example closed cell foam, with the adhesive exposed on the top surface, according to some embodiments;

FIG. 7A is a top, perspective view of the closed cell foam of FIG. 6 attached to the flexible circuit board of FIG. 5;

FIG. 7B is a top perspective view of four pieces of the closed cell foam of FIG. 6B attached to the flexible circuit board of FIG. 5;

FIG. 8A is a top, perspective view of an example rectangular ultrasound transducer, according to some embodiments;

FIG. 8B is a top, perspective view of an example flexible circuit board (FCB) finger, the view being of the side of the finger facing away from the transducer from FIG. 8A when wrapped around, according to some embodiments;

FIG. 8C is a top, perspective view of the flexible circuit board (FCB) finger from FIG. 8B, the view being of the side of the finger facing towards the transducer from FIG. 8A when wrapped around, according to some embodiments;

FIG. 8D is a cross section side view through the vertical plane defined by line BB′ of FIG. 8B, according to some embodiments;

FIG. 8E is a cross section side view through the vertical plane defined by line CC′ of FIG. 8B, according to some embodiments;

FIG. 8F is a bottom, perspective view of the rectangular ultrasound transducer of FIG. 8A with the flexible circuit board finger of FIG. 8B conductively attached to a back electrode of the transducer;

FIG. 8G is a top, perspective view of the rectangular ultrasound transducer of FIG. 8A with the flexible circuit board finger of FIG. 8B electrically attached to a front electrode of the transducer;

FIG. 9A is a top, perspective view of an example flat ultrasound transducer array, according to some embodiments;

FIG. 9B is a cross section side view through the vertical plane defined by line DD′ of FIG. 9A, according to embodiments;

FIG. 9C is a top perspective view of another example flat ultrasound transducer array, according to embodiments;

FIG. 9D is a cross section view through the vertical plane defined by line FF of FIG. 9C, according to embodiments;

FIG. 10 is a top perspective view of an example curved ultrasound transducer array of FIG. 9A, according to some embodiments; and

FIG. 11 is a top, perspective view of the curved flexible array of FIG. 10, shown encapsulated in a flexible material.

### DETAILED DESCRIPTION OF EMBODIMENTS

Reference will now be made to the accompanying drawings, which assist in illustrating the various pertinent features of the present system. The following description is presented for purposes of illustration and description and is not intended to limit the inventions to the forms disclosed herein. Consequently, variations and modifications commensurate with the following teachings, and skill and knowledge of the relevant art, are within the scope of the presented embodiments. The embodiments described herein are further intended to explain the best modes known of practicing the inventions and to enable others skilled in the art to utilize the inventions in such, or other embodiments and with various modifications required by the particular application(s) or use(s) of the presented inventions.

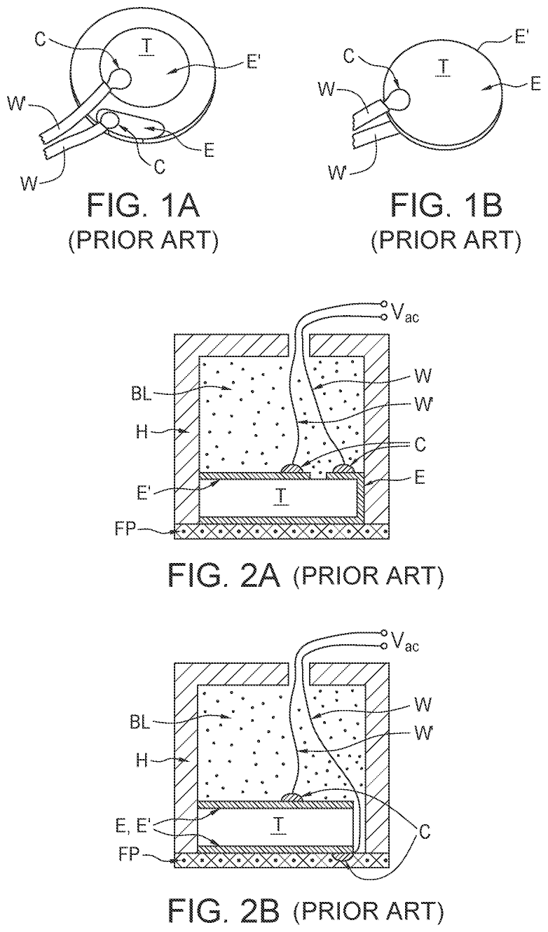

By way of background, known prior art intraoral ultrasound devices will first be described having regard to FIGS. 1 and 2.

FIG. 1A (PRIOR ART) provides a schematic representation of an example bare piezoelectric ultrasound transducer T used to emit ultrasound in known ultrasound devices. Generally, the transducer T has a wrap-around electrode E (meaning an electrode that is accessible from one side of the transducer T but allows a connection to the other side) and a central electrode E′. A gap is typically formed between the wrap-around and central electrodes E,E′, the gap being equal to or greater than the thickness of the transducer T, and electrical wires W, W′ are used to electrically connect the transducer T to operating componentry (not shown).

In such an example device, transducer T may be substantially circular in shape and may have an external diameter in the order of a few centimeters. Transducer T may consist of a piezoelectric (PZT) transducer (e.g., lead zirconate titanate), and may have an approximate thickness of 1.4 mm (representing half of the wavelength of the resonant frequency of 1.5 MHz in the PZT piezoelectric material). The wrap-around and central electrodes E,E′ may be manufactured from silver, or any other suitable material known in the art.

Electrical wires W,W′ corresponding to each of the wrap-around and central electrodes E,E′, respectively, may be rigidly connected (e.g. soldered) on the same side of the transducer T, creating at least two soldered joints or connections C between the wires and the electrodes. Alternatively, electrical wires W,W′ may be rigidly connected to electrodes E,E′ by other means, such as by using a conductive epoxy.

FIG. 1B (PRIOR ART) provides a schematic representation of another example bare piezoelectric ultrasound transducer T used in known ultrasound devices. Generally, the transducer T is again substantially circular in shape, having an external diameter in the order of a few centimeters, and again having an approximate thickness of 1.4 mm (which represents half of the wavelength of the resonant frequency of 1.5 MHz in the PZT piezoelectric material). In this example, however, the device comprises electrodes E,E′ positioned on opposite sides of the transducer T and, as a result, the corresponding electrical wires W,W′ must be rigidly connected (i.e. soldered) to electrodes E,E′, respectively, on opposed sides of the transducer T.

Both of the foregoing background examples are illustrated to demonstrate rigidly soldered connections between electrical wires and the electrodes of transducers. As would be appreciated by those skilled in the art, such mechanical attachment of the electrodes (located under the solder joints) and the transducer PZT material are mechanically weak areas, particularly when repeated flexing or pulling on the wires during use may result in the electrodes (located under the solder joints) detaching from the transducer and leading to a non-functional transducer. As a result, known ultrasound devices having soldered or other rigid connections between the electrical wires and the electrodes of the transducers frequently suffer from detachment of the electrodes, leading to a non-functional device.

Attempts have been made to minimize the drawbacks of ultrasound devices having transducers with soldered connections. FIG. 2A (PRIOR ART) provides a schematic representation of such an improvement whereby the transducer T (from FIG. 1A) is ‘protected’ by encapsulating it within a stiff housing H. The housing H is typically comprised of a rigid material, such as metal or plastic, and may further consist of a front wear plate FP (also called a matching layer) and a backing layer BL.

In the foregoing example, the backing layer BL is typically comprised of a low-density material, such as foam or air, or other known materials suitable for ultrasound reflection. That is, the backing layer BL may be manufactured from any low-density material appropriate for use where the transducer T is to be optimized for continuous emitting. However, backing layer BL may alternatively be made of ultrasound absorbing material with high density, for example, if the transducer T is optimized for emitting short pulses and sensing return waves (echoes). The front wear plate FP may be approximately a quarter wavelength thick and may function as acoustic impedance matching layer. That is, front wear plate FP is typically comprised of a material exhibiting small losses to ultrasound propagation, for example, plastics, silicones, or epoxies.

The electrical wires W,W′ are again soldered to the wrap-around and central electrodes E,E′, as above, creating two solder joints or connections C where the wires W,W′ attach to the electrodes E,E′. In contrast to the previous examples, however, the wires W,W′ exit housing H for connection to an output signal of an ultrasound transducer driver AC voltage Vac and/or the input of a sensing circuitry the connections C, such that the housing H serves to protect connections C from being pulled, flexed, or detached.

FIG. 2B (PRIOR ART) provides a schematic representation of the transducer T shown in FIG. 1B encapsulated within a rigid housing H, as above. Housing H again has a front wear plate FP (also called a matching layer) and a backing layer BL. As in the previous example, electrical wires W,W′ are soldered to the electrodes E,E′, respectively, but on opposed sides of the transducer T, thereby creating two soldered connections C where the wires W,W′ attach to the electrodes E,E′. As above, electrical wires E,E′ may exit the external housing H and may be connected to the output signal of an ultrasound transducer driver AC voltage Vac and/or the input of a sensing circuitry.

Both of the foregoing examples shown in FIGS. 2A and 2B are again illustrated to demonstrate the use of a rigid housing H as a means for minimizing damage to and/or disconnection of the connections C. Positioning the connections C between the wires W,W′ and the electrodes E,E′, respectively, ensures that only the portion of the wires W,W′ outside of housing H can bend and prevent the soldered connection C from being flexed. As a result, these example devices are able to protect weak points, such as connections C, reducing electrical connectivity problems that existed in earlier devices (i.e. where disconnection resulting in a non-functional transducer T). Unfortunately, however, use of the cumbersome, large and rigid housing H is not suitable for small, flexible arrays where the housing H would increase the size of the array and/or would reduce the array flexibility.

In light of the foregoing background examples, there remains a clear need for an improved intraoral ultrasound therapy device having improved electrical connections capable of withstanding maximum forces applied to the connections C, without cracking or breaking when the connections C become deformed.

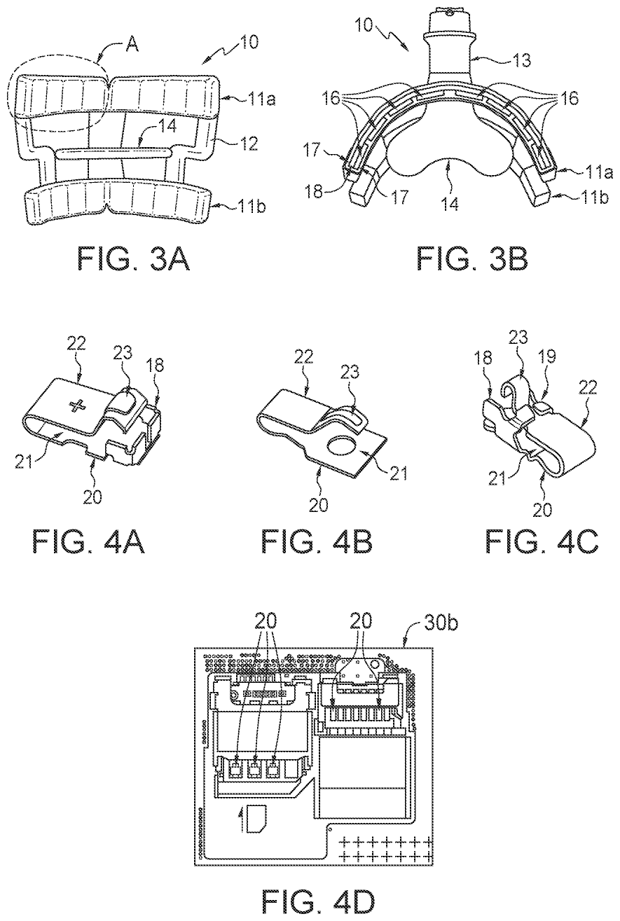

Broadly, according to embodiments, an improved intraoral ultrasound apparatus 10 and methodologies of use are provided, the apparatus 10 generally configured to have flexible electrical connections. As will be described in more detail, the presently improved ultrasound-emitting apparatus 10 may comprise at least one ultrasound transducer(s) or array of transducers, for emitting at least one ultrasound emission, a flexible printed circuit board, and at least one flexible electrical connection between the at least one ultrasound transducer and the flexible printed circuit board. The ultrasound-emitting apparatus 10 may be encapsulated or housed within a flexible mouthpiece for improved intraoral therapy and may be operably connected to an electronics controller (as would be known in the art). The subject apparatus 10 and methodologies of use will now be described with specific reference to FIGS. 3-11.

FIG. 3A provides a schematic representation of the present apparatus 10 encapsulated or housed within a flexible and adjustable mouthpiece for improved intraoral therapy. For example, without limitation, the present apparatus 10 may be housed within a flexible and adjustable mouthpiece, such as that described in International Patent Application No. PCT/CA2019/051234, incorporated herein by reference in its entirety, wherein the mouthpiece houses an upper panel 11a of ultrasound transducer(s) 16 and a lower panel 11b of ultrasound transducer(s) 16, and at least one adjustable connector 12 interconnecting the upper and lower panels 11a,11b. The mouthpiece may comprise a neck 13 for attaching the mouthpiece to an enclosure with an electronics controller (not shown). In some embodiments, the neck 13 may also allow for the extension of the flexible circuit board from inside the arrays 11a,11b, to electrically connect to the electronics controller. The mouthpiece may further comprise a bite plate 14 for use by the patient to hold the mouthpiece in place within the patient's mouth (i.e. the patient can bite down on the plate to maintain the apparatus 10 in position).

FIG. 3B provides a schematic cross-sectional representation of the upper transducer array panel 11a, such that the positioning the at least one transducer(s) 16 within the panel 11a are visible. While the upper panel 11a is shown, it should be appreciated that the at least one transducer(s) 16 of lower panel 11b may be similarly positioned to those depicted in the upper panel 11a.

In some embodiments, each upper and lower array panel 11a,11b may comprise one or more transducer(s) 16, and preferably at least eight transducers (16; FIG. 3B). The transducers 16 may be bulk (thickness mode) piezoceramic transducers driven at or close to resonance (that is, a thickness of half a wavelength). Each at least one transducer 16 may have a dimension (e.g. diameter, or width and length) of less than ten wavelengths of the ultrasound in the piezoelectric material from which the transducer 16 is made. That is, for PZT material at 1.5 MHz, the diameter, or width and length, of the transducer(s) 16 may be less than approximately 28 mm. The transducer(s) 16 may be circular, square, rectangular, or any other suitable shape as would be known in the art.

The mouthpiece for receiving and housing the at least one transducer(s) 16 may consist of a flexible material 17 for encapsulating the internal components of the apparatus including, without limitation, the transducer(s) 16, the flexible circuit board (not visible in FIG. 3B), and at least a layer of backing material 18 operably connecting the transducers 16 to the flexible circuit board 30. That is, in some embodiments, each of the at least one transducer 16 may be connected, via a flexible transducer backing material 18, to the flexible printed circuit board 30. In this regard, the presently improved apparatus 10, housed within flexible mouthpiece material 17, may advantageously be formed into a flat or a curved configuration (i.e. for ease of use within a patient's mouth).

The flexible housing material 17 may consist of any appropriately flexible material 17 including, without limitation, a silicone elastomer or silicone rubber. In some embodiments, the flexible material 17 may comprise a biocompatible material such as silicone elastomer MED-6033, liquid silicone rubbers MED-4950, MED-4940, MED-4930, or the like.

The transducer backing material 18 may be positioned in between, and serve to attach, the transducers 16 and the printed circuit board 30. In some embodiments, backing material 18 may consist of air, or a low acoustic impedance material. In other embodiments, the backing material 18 may consist of, without limitation, a closed cell foam material.

As will now be described in more detail, the flexible materials used to house and operably connect components of the present apparatus 10 enable the apparatus to be specifically configured for providing a soft or flexible connections between the at least one transducer(s) 16 the flexible circuit board 30 in each array 11a,11b, eliminating the need for rigid (e.g. soldered) connections. Advantageously, such flexible connections allow for vertical and/or horizontal displacement and/or tilting of the at least one transducer(s) 16 relative to the circuit board 30, while maintaining electrical connectivity therebetween. Such flexible connections also allow for temporary disconnection of the electrical connectivity between components when an excessive deformation force is applied, and for self-reconnecting between components following the removal of the excessive deformation (i.e. providing blind mating of electrical contacts when the transducers are coupled to the flexible circuit board).

According to embodiments, having regard to FIGS. 4A-D, the present apparatus 10 may be configured to provide at least one soft or flexible connection between the at least one transducer(s) 16 and the printed circuit board 30, such connections consisting of, for example, one or more flexible spring-biased contacts 20. As will be described, spring contacts 20 provide compressible, spring-loaded connection or electrical contact points between the at least one transducer(s) 16 and the flexible circuit board 30. Herein, spring contacts 20 may also be referred to as spring fingers or C-clip connectors.

FIGS. 4A, 4B and 4C, provide schematic representation of example spring contacts 20. According to embodiments, example spring-loaded connectors 20 may consist of a base portion 21 and a cantilevered arm portion 22, wherein, at a first end, arm 22 may extend upwardly and be biased away from base 21. At a second end, arm 22 may support at least one electrical contact point, line, or surface 23, such contact point 23 for making an electrical connection between the at least one transducer(s) 16 and the circuit board 30. Base portion 21 may be used to attach spring contacts 20 to the flexible circuit board 30. For example, in some embodiments, base 21 may form a mounting plate for securely affixing spring contacts 20 to the flexible circuit board 30. It would be understood that any appropriate means for affixing spring contacts 20 to flexible circuit board 30 are contemplated including, without limitation, soldering the contacts 20 to the printed flexible circuit board 30.

In some embodiments, having regard to FIGS. 4A and 4C, spring contacts 20 may further comprise a stop 24 for controllably stopping the compression or biasing of arm 22 towards base 21. That is, when transducer(s) 16 is electrically coupled to electrical contact point 23, arm 22 may be compressed or biased downwardly towards base 21 until transducer back side 16b (not visible in FIGS. 4A and 4C shown) abuts compression stop 24. As would be understood, when no transducer(s) 16 is coupled, arm 22 may be fully extended (biased upwardly) away from base 21. Having further regard to FIG. 4C, each at least one spring contact 20 may further comprise a deflection stop 25. The deflection stop 25 restricts the arm 22 from lifting further then allowed by the deflection stop 25. The deflection stop 25 allows for the arm 22 to have a preset tension larger than zero in its uncompressed position or state.

According to embodiments, the at least one spring contacts 20 may be any suitable spring contacts known in the art including, but not limited to, spring contacts 57131-45R, 57221-45R, 57241-45R, 57251-45R and 57261-45R (Harwin Inc, Indiana, USA), C-Clip Connector Part Number W9908 (Pulse Electronics, Pennsylvania, USA), and/or spring finger drawing number C-2199248 (TE Connectivity, Pennsylvania, USA). Any adaptation or modification of the present spring contact 20 may be used to achieve the desired result.

FIG. 4D provides a schematic representation of an example rigid printed circuit board 30b having a plurality of spring contacts or connectors 20 mounted thereon. As would be understood by those skilled in the art, according to embodiments, rigid printed circuit board 30b may be similar to the printed circuit boards used in cellphones used to provide flexible connectivity to an SD (Secure Digital) card and a SIM (Subscriber Identification Module) card.

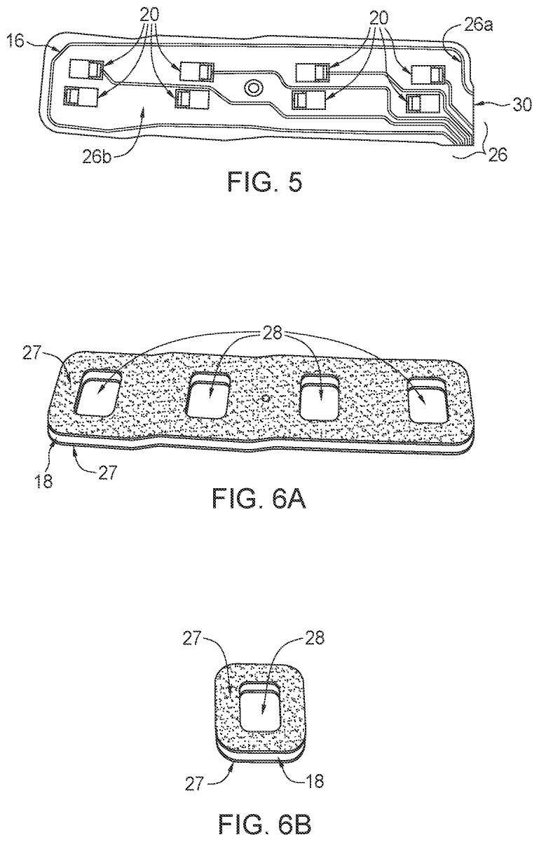

FIG. 5 provides a schematic isolated side view of a half of a flexible printed circuit board 30 of upper transducer panel 11a shown in FIG. 3A (circle A), the half panel 11a having at least four transducer(s) 16 operably connected to at least eight (four pairs) spring contacts 20. For example, each at least one transducer(s) 16 may be electrically coupled to flexible circuit board 30 by at least one pair, i.e. two, spring contacts 20. Circuit traces 26 may connect each at least one spring contact 20 to an electronics controller (not shown). Each spring contact pair has one of the springs 20 electrically connected to the electronics controller (not shown) through a circuit trace 26, and the second spring contact 20 directly connected to a ground plane 26b located underneath the spring contact base portion 21, where the ground plane 26b further connects to the electronics controller (not shown) It should be appreciated that although only half of upper panel array 11a is shown, each of the other upper half panel array 11a and the lower array 11b of the apparatus may be similarly configured.

In addition, FIG. 5 depicts a perimeter circuit trace 26a that follows the contour/edge of the flexible circuit board 30 and connects to the electronics controller. Perimeter trace 26a may serve to detect if/when the flexible circuit board 30 rips during manufacturing, or when in use, due to excessive force. That is, where the perimeter trace 26a becomes interrupted (open circuit), i.e. where circuit board 30 rips from the edges, the interruption can be detected automatically by the electronics controller or manually measured by an operator during manufacturing or servicing.

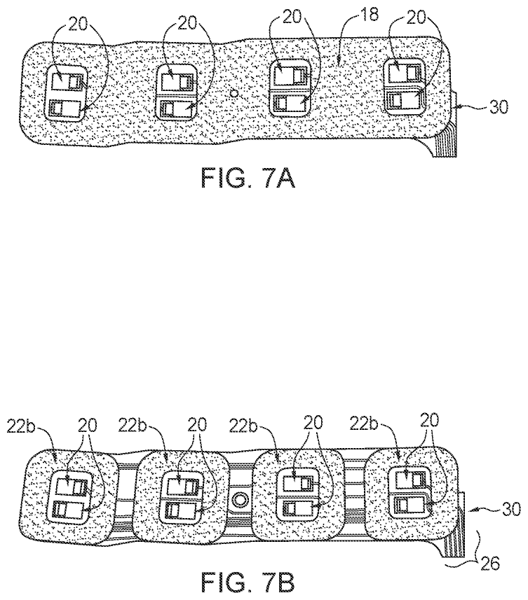

FIG. 6A depicts a schematic representation of a solid piece of backing layer 18 that may be used to attach the at least one transducer(s) 16 to the printed flexible circuit board 30 and its corresponding spring contacts 20. That is, backing layer 18 may be adhered on one side to flexible circuit board 30 and on the other side to the at least one transducer(s) 16. When each of the at least one transducer(s) 16 are adhered to backing layer 18, the corresponding spring contacts 20 of flexible circuit board 30 are received and accommodated within backing cutouts or slots 28 (e.g. as shown in FIG. 7A). In this regard, for example, each of the top and bottom surfaces of backing layer 18 may comprise at least one layer of adhesive material for attaching the material 18 to the transducers 16 and the flexible circuit board 30, respectively. In some embodiments, the adhesive layer may comprise a layer of adhesive transfer tape 27, or the like, such adhesive transfer tape 27 having a liner than may be peeled away to expose the adhesive layer underneath. The liner may be a poly-coated kraft liner or any other suitable liner material. In some embodiments, adhesive layer 27 may be manufactured from an acrylic based adhesive, such as 3M 467MP and 3M 468MP. In other embodiments the adhesive layer 27 may be applied to the backing layer 18 through other methods such as dispensing, screen printing, or spraying. Furthermore, the adhesive layer 27 can be applied selectively (while avoiding covering any electrical contacts) to the flexible circuit board 30 and to the back side 16b of transducers 16.

FIG. 6B depicts a schematic representation of an individual piece of backing layer 18 for use in attaching an individual at least one transducer(s) 16 to the printed flexible circuit board 30. As above, each top and bottom surfaces of the backing layer 18 may have at least one or more layers of adhesive or transfer tape 27, or the like, for securing backing layer 18 to both the flexible circuit board 30 on one side and to at least one transducer(s) 16 on the opposite side, while accommodating spring contacts 20 within slot 28.

According to embodiments, backing layer 18 and adhesive layer 27 are each made of suitable materials to withstand the high temperatures used in subsequent encapsulation steps as described below. In some embodiments, as above, backing layer 18 may be manufactured from closed cell foam, such as a nylon-based foam, polyurethane foam, or silicone foam. In other embodiments, backing layer 18 and adhesive layer 27 may be any other suitable materials, or may be only air within the flexible encapsulating material 17 on opposed sides of each transducer(s) 16. Any adaptation or modification of the present backing layer 18 and/or adhesive layer 27 may be used to achieve the desired result.

As above FIG. 7A provides a schematic representation of backing layer 18 positioned on a flexible circuit board 30, such that each pair of spring contacts 20 coupled to the transducer(s) 16 are received and protected within slots 28. FIG. 7B provides a schematic representation of individual sections or pieces of backing layer 18 applied to flexible circuit board 30, wherein the liner protecting the adhesive transfer tape 27 on both sides of backing layer 18 has been removed to allow adhesion of the backing layer 18 to the flexible circuit board 30 and of the transducers 16 to the backing layer 18.

In use, when the backing layer 18 is applied to the flexible circuit board 30, the at least one slots 28 in layer 18 may be substantially aligned with the spring contacts 20. As would be appreciated, slots 28 may be sized and shaped to receive spring contacts 20 therein, the slots 28 preferably being sufficiently large so as not to interfere with the spring contacts 20 while also being as small as possible to maximize the surface area (i.e. the adhesive interface) between backing layer 18 with both the flexible circuit board 30 and the transducer(s) 16.

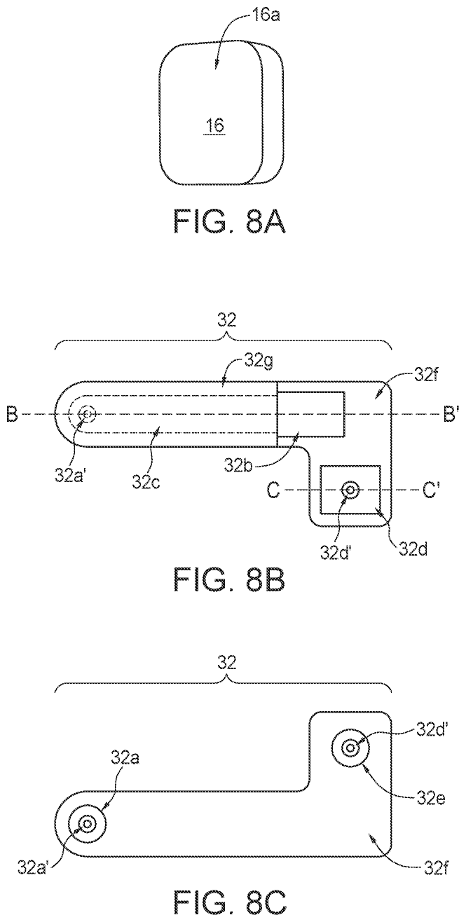

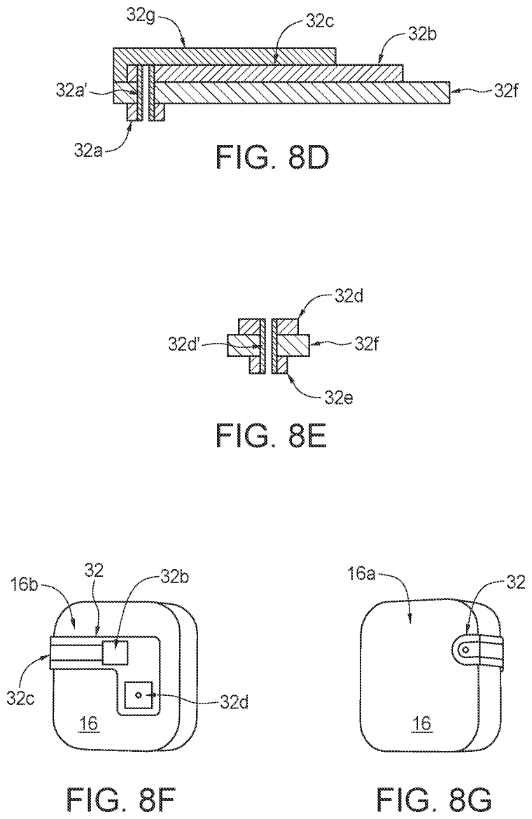

FIGS. 8A, 8F and 8G show an example of the at least one ultrasound transducer(s) 16 in more detail. Transducer(s) 16 may comprise a rectangular piezoelectric transducer, and may be made of PZT or any other suitable piezoelectric material. In some embodiments, the at least one transducer(s) 16 may be approximately 8 mm×9 mm with rounded corners, and may be approximately 1.4 mm thick, or any other size and configuration as may be appropriate. In other embodiments, the at least one transducer(s) 16 may be larger in size such as, for example, up to at least 15 mm in diameter or width. In some embodiments, the transducer 16 may be any other suitable ultrasound transducer, such at least one transducer(s) 16 capable of resonating at a 1.5 MHz drive signal.

In some embodiments, transducer(s) 16 may comprise a front electrode adhered to the front surface of the transducer 16a and a back electrode adhered to the opposite or back side of the transducer 16b (e.g. a front electrode is shown on surface 16a in FIGS. 8A and 8G, while a back electrode is shown on surface 16b of FIG. 8F). For example, as will be described, front and back electrodes may consist of a flexible ‘finger’ element. FIGS. 8B to 8E show an example flexible circuit board ‘finger’ element 32, the finger 32 operative to provide an electrical connection between at least one of the transducer(s) 16 and its corresponding pair of spring contacts 20.

More specifically, FIG. 8B shows the top, perspective front view of the flexible circuit board (FCB) finger 32, the view being of the side of the finger 32 facing away from the transducer 16 from FIG. 8A when wrapped around the transducer 16. The flexible circuit board finger 32 may have two contact points, or pads, a first front rectangular pad 32b and a second back circular pad 32a (not visible in FIG. 8B, but shown in FIGS. 8C and 8D), the front and back pads 32b,32a being connected to one another by conductive trace 32c and conductive via 32a′ (the via 32a′ is round and is located in the center of the back circular pad 32a). The finger 32 may further comprise a front rectangular pad 32d, where the top pad 32d is electrically connected through a conductive via 32d′ to a back circular pad 32e (not visible in FIG. 8B, but shown in FIGS. 8C and 8E).

FIG. 8C shows the top, perspective back view of the flexible circuit board finger 32, the view being of the side of the finger 32 facing towards transducer 16 from FIG. 8A when wrapped around transducer 16. In addition, to the back circular pads 32a and 32e, and vias 32a′ and 32d′, FIG. 8C also shows the insulating substrate 32f of flexible circuit board finger 32.

FIG. 8D shows a cross section side view through the vertical plane defined by line BB′ of FIG. 8B, according to some embodiments. The front rectangular pad 32b is part of the trace 32c, and through the via 32a′ connects to the back circular pad 32a. The insulator substrate 32f of the FCB finger 32 is shown. Insulating front cover lay 32g is also illustrated.

FIG. 8E shows a cross section side view through the vertical plane defined by line CC′ of FIG. 8B. The front rectangular pad 32d is connected through the via 32d′ to the back circular pad 32e. The insulator substrate 32f of the FCB finger 32 is also shown.

According to embodiments, having regard to FIG. 8F, the flexible circuit board finger 32 may be secured to the back surface 16b of a transducer 16. For example, the finger element 32 may be secured in place using any appropriate attachment means including, without limitation, conductive epoxy, soldering, conductive tape, or any other suitable attachment means.

Regarding FIG. 8F, in some embodiments, pads 32b and 32d may be positioned on the back surface 16b of transducer 16, such that pad 32d may be electrically connected (through via 32d′ and back pad 32e) to the back surface electrode 16b of transducer 16 and such that each pad 32b,32d align with one of a corresponding pair of spring contacts 20 on the flexible circuit board 30 (as will be described in more detail below). That is, pads 32b,32d may be positioned adjacent one another on transducer 16 so as to each provide a respective contact point with one of the spring contacts 20 (i.e. contact may arise between the approximate center of pads 32b,32d and springs 20 in an undeformed array state, such state being, for example, a state in which no force or deformation is applied). In other embodiments, pad 32d may be omitted and at least one electrical spring contact point 23 may make contact directly with the transducer back electrode 16b.

Regarding FIG. 8G, in some embodiments, pad 32a may be positioned on the front surface 16a of transducer 16 and electrically connected to back pad 32b. In this regard, advantageously, both front and back electrodes adhered to the transducer 16 may be accessible from the same side without the undesired Effective Radiating Area (ERA) reduction that may be observed in conventional transducers using wrap-around electrode configurations.

For example, it is contemplated that the at least one transducer(s) 16 may comprise a wrap-around electrode (not shown), similar to the wrap-around electrodes known and used in the prior art (e.g. FIG. 1A). In such cases, embodiments may comprise a gap of at least 1.4 mm between the wrap-around electrode and a central electrode (not shown), where the transducer area under the wrap-around electrode and under the gap is not an active area and does not emit longitudinal ultrasound waves, such area being considered a sacrificial area for the convenience of having access to both electrodes on both sides. That is, if the transducer is too small (i.e. having a diameter or width of less than about 15 mm), having a wrap-around electrode may result in too large of a portion of the transducer surface being non-emitting, reducing the ERA of that transducer. Accordingly, herein, such an embodiment may not be suitable for all compact medical applications.

It is further contemplated that any other suitable electrical contact may be used to electrically connect one of the transducer(s) 16 to a respective, corresponding pair of spring contacts 20.

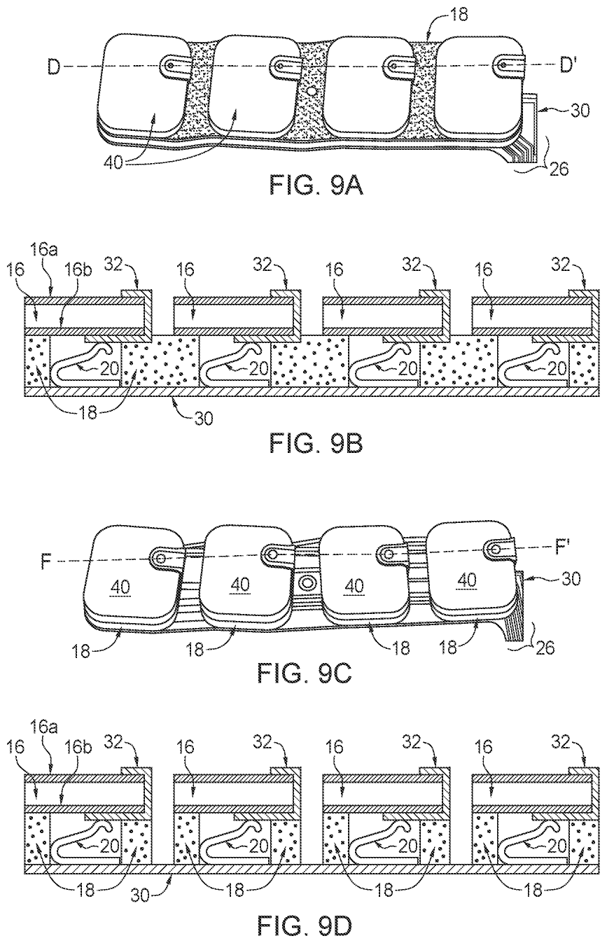

Herein, the combination of the at least one transducer(s) 16 and the flexible circuit board finger portion 32 will hereinafter be referred to collectively as a compact transducer assembly 40. FIG. 9A shows an example of a flexible ultrasound transducer array in a flat position, the flat ultrasound transducer array having four compact transducer assemblies 40 attached to the backing layer 18 by the adhesive transfer tape 27. Each compact transducer assembly 40 may be in electrical contact with a respective pair of spring contacts 20 surface mounted on the flexible circuit board 30 (spring contacts 20 are not visible in FIG. 9A as they are obscured by the transducer assemblies 40). In some embodiments, the flexible printed circuit 30 may be stiffened behind each transducer 16 to help maintain contact between the spring contacts 20 and the pads 32b and 32d of FCB finger 32 adhered to the transducer(s) 16.

FIG. 9B shows an example cross-sectional view of the flexible circuit board 30 (along line DD′; FIG. 9A), the view depicting four pairs spring contacts 20 attached to flexible circuit board 30. More specifically, FIG. 9B depicts the connection between the four pairs of spring contacts 20 and the corresponding four flexible circuit board fingers 32, each finger element 32 being wrapped around an ultrasound transducer(s) 16. Each at least one transducer(s) 16 is attached to backing layer 18 which is in turn attached to the flexible circuit board 30.

FIG. 9C shows an example perspective view of an alternative embodiment of the present flexible ultrasound transducer array 30, the array again being depicted laid out in a flat position. According to embodiments, the array 30 comprises four compact transducer assemblies 40 attached to four separate pieces of backing material 18 by adhesive transfer tape 27. Each compact transducer assembly 40 may be in electrical contact with a respective pair of spring contacts 20 surface mounted on the flexible circuit board 30 (spring contacts 20 are not visible in FIG. 9C as they are obscured by the transducer assemblies 40). Advantageously, embodiments utilizing individual pieces of backing layer 18 may having increased flexibility of the encapsulated array assemblies 40, as compared to using a continuous piece of back layer 18 for multiple transducers 16, e.g. FIG. 9A above).

FIG. 9D shows an example cross-sectional view of the flexible circuit board 30 (along line FF′; FIG. 9C), the view depicting four pairs of spring contacts 20 attached to flexible circuit board 30. More specifically, FIG. 9D depicts the connection between the four pairs of spring contacts 20 and the corresponding four flexible circuit board fingers 32, each finger element 32 being wrapped around an ultrasound transducer(s) 16. Each at least one transducer(s) 16 is attached to an individual piece of backing layer 18, each piece of baking layer 18 in turn being attached to the flexible circuit board 30.

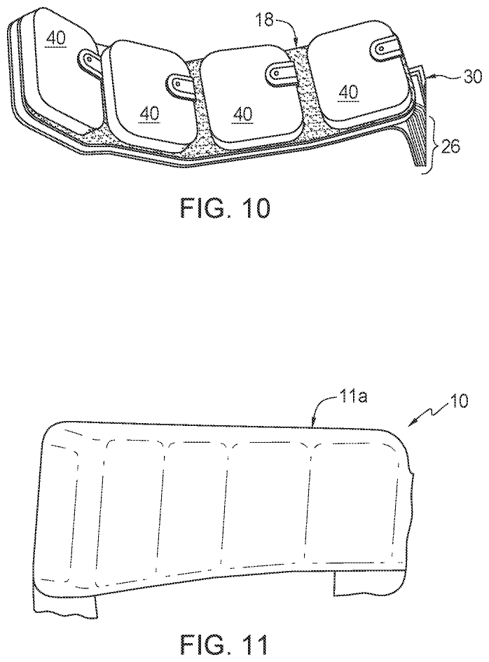

According to embodiments, the present flexible ultrasound transducer array may be positioned in a curved position for ease of use. Having regard to FIG. 10, the curved ultrasound transducer array comprises four compact transducers assemblies 40 attached to the backing layer 18 by the adhesive transfer tape 27. Each compact transducer assembly 40 may be in electrical contact with a respective pair of spring contacts 20 surface mounted on the flexible circuit board 30 (spring contacts 20 are not visible in FIG. 10 as they are obscured by the transducer assemblies 40). It is an advantage of the of presently curved array that the array may flex to form a concave, flat, convex, or twisted shape as needed in the field or during manufacturing or testing, while maintaining its electrical connectivity integrity.

In some embodiments, each compact transducer assembly 40 is vertically positioned over the respective pair of spring contacts 20 such that the pads 32b,32d of the finger 32 are in contact with the electrical contact points 23 of the spring contacts 20 and the flexible arm 22 is at least partially compressed. In some embodiments, the flexible arm 22 is approximately halfway compressed. In some embodiments, the vertical positioning of the compact transducer assembly 40 may be determined by the thickness of the backing layer 18. In some embodiments, the thickness of the backing layer 18 can be reduced if necessary by compressing the backing layer 18 by pressing on the transducer assembly 40 and while heating the transducer assembly 40 in order to transfer the heat to the backing layer 18 and to determine localized memory loss of the backing layer 18 and therefore reduce its thickness to a desired level for the position of the contact point 23. In some embodiments, the vertical positioning of the compact transducer assembly 40 may be determined by the shape and dimensions of a flexible encapsulating layer, as described below.

Therefore, in some embodiments, each transducer assembly 40 may stay in blind contact with the contact points 23 of the respective pair of spring contacts 20 when the transducer assembly 40 is vertically displaced within the flexing range of the flexible arm 22, which is in the order of approximately +/−0.3 mm in this embodiment. Therefore, in some embodiments, the transducer assemblies 40 are able to stay electrically connected to the flexible circuit board 30 when the transducer assemblies 40 are vertically displaced with respect to the flexible circuit board 30 due to an external deformation force.

Each transducer assembly 40 may also stay in contact with the electrical contact points 23 of the respective pair of spring contacts 20 when the transducer assembly 40 is horizontally displaced within approximately half the width of the pads 32b,32d of the finger 32, which in this example is in the order of +/−1 mm. In some embodiments, the mechanical loading of the contact point 23 on the transducer assembly 40 may be minimal, and as a result the efficiency and resonant frequency of the transducer 40 is minimally affected. Therefore, in some embodiments, the transducer assemblies 40 are able to stay electrically connected to the flexible circuit board 32 when the transducer assemblies 40 are horizontally displaced with respect to the flexible circuit board 30 due to an external deformation force.

FIG. 11 shows the curved ultrasound transducer array of FIG. 10, the array being encapsulated in a thin layer of flexible material 17 to form an encapsulated array portion of the present intraoral therapy device 10 (box A in FIG. 3A). In some embodiments, the layer of flexible material 17 is thinner than the transducer 16 thickness. In some embodiments, the flexible material 17 may comprise silicone elastomer, silicone rubber, or any other suitable flexible material. The transducer array may be molded in the flexible material 17 and the flexible material 17 may be cured at a high temperature. Encapsulation in the flexible material 17 may allow the transducer assemblies 40 to be secured in the undeformed state in a flexible manner such that the transducer assemblies 40 may flex back to the undeformed state after the external deformation force is removed.

Herein, in use, methods for providing intraoral ultrasound therapy are provided, the methods comprising providing at least one ultrasound transducer for emitting at least one ultrasound emission; providing a printed flexible circuit board; connecting the at least one ultrasound transducer to the flexible printed circuit board by a flexible electrical connection, and administering the at least one ultrasound emission to a patient. The methods may include providing a flexible electrical connection comprising at least one spring contact.

In some embodiments, if the displacement of the transducer assembly 40 due to an external deformation force exceeds the vertical and horizontal limits described above, the transducer assemblies 40 may become temporarily electrically disconnected from the spring contacts 20 until the deformation force is removed. Once the deformation force is removed, the elastic forces of the flexible encapsulation material 17 may reposition the transducer assemblies 40 back to their undeformed position and electrical connectivity may be regained.

The encapsulated array portion 40 shown in FIG. 11 may correspond to the portion of the upper flexible array 11a indicated in circle A of FIG. 3A. The other half of the upper array 11a, and the two halves of the lower array 11b, may be similar in structure to the encapsulated array portion 40 of FIG. 11. The mouthpiece 10 may thereby demonstrate high electrical reliability and strength in the field.

Although certain embodiments describe herein provide for the use of flexible circuit boards, in other embodiments, the present apparatus 10 could also be applied to a rigid printed circuit board, allowing the top surface of the encapsulated transducers to move despite the rigid printed circuit board therebeneath.

According to embodiments, methods of making an improved ultrasound apparatus are provided. The present methodologies may be used to make or manufacture the present apparatus 10 as described above.

Herein, the present methods may comprise providing at least one ultrasound transducer(s) 16 and a flexible printed circuit board 30, wherein the at least one ultrasound transducer(s) 16 may be electrically connected to the flexible circuit board 30 by a flexible or soft electrical connection. In some embodiments, the flexible electrical connection may comprise at least one spring contact 20 electrically coupled to at least one electrode finger element 32.

In some embodiments, providing at least one ultrasound transducer comprises providing an array of ultrasound transducers. In some embodiments, the array of ultrasound transducers is a flexible array.

In some embodiments, the method further comprises encapsulating the array of ultrasound transducers in a flexible material. In some embodiments, the flexible material is silicone elastomer, silicone rubber, or any other suitable flexible material. In some embodiments, encapsulating the array of ultrasound transducers further comprises molding the array in the flexible material and curing the flexible material at a high temperature.

Various modifications besides those already described are possible without departing from the concepts disclosed herein. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly reference.

Although particular embodiments have been shown and described, it will be appreciated by those skilled in the art that various changes and modifications might be made without departing from the scope of the disclosure. The terms and expressions used in the preceding specification have been used herein as terms of description and not of limitation, and there is no intention in the use of such terms and expressions of excluding equivalents of the features shown and described or portions thereof.

1. An ultrasound apparatus, the apparatus comprising:
at least one ultrasound transducer, for emitting at least one ultrasound emission,
a printed circuit board, and
a flexible electrical connection between the at least one ultrasound transducer and the printed circuit board.

2. The apparatus of claim 1, wherein the apparatus comprises at least one array of ultrasound transducers.

3. The apparatus of claim 2, wherein each at least one array of ultrasound transducers comprises at least eight ultrasound transducers.

4. The apparatus of any one of claim 1, wherein the at least one transducers comprise piezoceramic ultrasound transducers.

5. The apparatus of claim 1, wherein the flexible electrical connection may comprise at least one spring contact.

6. The apparatus of claim 5, wherein the spring contact may comprise a mounting plate for securely affixing the contact to the circuit board.

7. The apparatus of claim 5, wherein the spring contact may comprise a cantilevered arm extending from the mounting plate for providing at least one electrical contact point.

8. The apparatus of claim 1, wherein the flexible electrical connection may further comprise at least one flexible circuit board finger for electrically coupling the at least one spring contact to the at least one ultrasound transducer.

9. The apparatus of claim 1, wherein the apparatus is encapsulated within a housing formed of flexible material.

10. The apparatus of claim 9, wherein the flexible housing material may comprise biocompatible silicone elastomer or silicone rubber.

11. The apparatus of claim 10, wherein the silicone elastomer may comprise MED-6033, or liquid silicone rubbers MED-4950, MED-4940, and MED-4930.

12. The apparatus of claim 1, wherein the apparatus further comprises at least one layer of transducer backing material operably connecting the at least one transducer(s) and the printed circuit board.

13. The apparatus of claim 12, wherein the backing material may comprise a closed cell foam.

14. The apparatus of claim 12, wherein the backing material may comprise nylon-based foam, polyurethane foam, or silicone foam.

15. The apparatus of claim 12, wherein the backing material may form at least one slot(s) for receiving and maintaining the at least one transducer(s).

16. A method for providing intraoral ultrasound therapy, the method comprising:
providing at least one ultrasound transducer for emitting at least one ultrasound emission;
providing a printed circuit board;
connecting the at least one ultrasound transducer to the printed circuit board by a flexible electrical connection, and
administering the at least one ultrasound emission to a patient.

17. The method of claim 16, wherein the flexible electrical connection comprises at least one spring contact.

18. The method of claim 16, wherein providing the at least one ultrasound transducer comprises providing a flexible array of ultrasound transducers.

19. The method of claim 18, further comprising encapsulating the flexible array of ultrasound transducers in a flexible material.

20. The method of claim 16, wherein the flexible material secures the flexible array of ultrasound transducers in an undeformed state.
